(19) United States
(12) Patent Application Publication
(10) Pub. No.: US 2022/0242917 A1
(43) Pub. Date: Aug. 4, 2022

(54) COMPOSITIONS AND METHODS FOR PRODUCING ADENO-ASSOCIATED VIRAL VECTORS

(71) Applicant: RootPath Genomics, Inc., Watertown, MA (US)

(72) Inventors: Xi CHEN, Newton, MA (US); Ely PORTER, Medford, MA (US)

(21) Appl. No.: 17/680,479

(22) Filed: Feb. 25, 2022

Related U.S. Application Data
(63) Continuation of application No. PCT/US2020/047738, filed on Aug. 25, 2020.
(60) Provisional application No. 62/891,727, filed on Aug. 26, 2019.

(51) Int. Cl. C07K 14/005; C12N 7/00

(52) U.S. Cl. CPC C07K 14/005; C12N 7/00; C12N 2800/107; C12N 2750/14123; C12N 2750/14122; C12N 2750/14152; C12N 2750/14143

(57) ABSTRACT

The present disclosure provides compositions and methods to make and use ribonucleic acid sequences encoding viral proteins (e.g., Rep and Cap proteins) derived from adeno-associated viruses. The RNA sequences can be delivered into a host cell for viral packaging.

### CROSS-REFERENCE

This application is a continuation of International Patent Application No. PCT/US2020/047738, filed Aug. 25, 2020, which claims the benefit of U.S. Provisional Patent Application No. 62/891,727, filed Aug. 26, 2019, each of which is incorporated herein by reference in its entirety.

### BACKGROUND OF THE INVENTION

Adeno-associated virus (AAV) was discovered in 1965, as a contaminant of adenovirus preparations. AAV has a linear single-stranded DNA (ssDNA) genome of approximately 4.7-kilobases (kb), with two 145 nucleotide-long inverted terminal repeats (ITRs) at the termini. The ITRs flank the two viral genes—rep (e.g., replication) and cap (e.g., capsid), encoding non-structural and structural proteins, respectively and can be useful for packaging of the AAV genome into the capsid and for initiating second strand DNA synthesis upon infection. AAV has been classified as a Dependoparvovirus (a genus in the Parvoviridae family) because it may require co-infection with helper viruses such as adenovirus, herpes simplex virus (HSV) or vaccinia virus for productive infection in cell culture (Atchison et al. (1965) Science 149:754; Buller et al. (1981) J Virol. 40: 241).

Recombinant AAV vector can be generated by replacing the wild-type AAV open reading frames with a transgene (e.g., a therapeutic or marker gene) expression cassette. When an AAV ITR-flanked transgene is present in cells that are expressing AAV Rep and Cap proteins, the Rep protein binds the ITRs flanking the transgene, initiating replication and producing single stranded copies of the ITR-flanked transgene. Rep then can guide the packaging of this ssDNA into assembling AAV capsids in the cell nucleus. Although there may be no overlapping sequence between the ITR-flanked gene therapy transgene and the AAV rep/cap gene, there may be the probability of generating wild-type replication competent AAV in which rep and cap have become inserted between the ITRs by non-homologous recombination (Allen et al., (1997) Journal of Virology 71: 6816-6822).

### SUMMARY OF THE INVENTION

Production of recombinant adeno-associated viral (AAV) particles may result in the production of wild-type AAV particles that may be impossible to separate from the gene therapy particles. Although AAV may not be associated with any diseases in humans and may have low immunogenicity, the presence of wild-type AAV particles can pose a safety concern.

Recognized herein is a need for compositions and methods for producing recombinant adeno-associated viral (AAV) particles that do not pose such safety concern. The compositions and methods provided herein can produce recombinant AAV particles that may not result in the production of wild-type and/or replication competent AAV particles.

In an aspect, the present disclosure provides a method for producing recombinant adeno-associated viral (AAV) particles, comprising delivering a ribonucleic acid (RNA) sequence encoding an AAV Replication (Rep) protein into a cell.

In some embodiments, the RNA sequence is a messenger RNA. In some embodiments, the AAV Rep protein is an AVV Rep78 protein or an AAV Rep 52 protein. In some embodiments, the AAV Rep protein comprises AVV Rep78 protein and AAV Rep52 protein. In some embodiments, the RNA sequence comprises a first RNA sequence encoding the AVV Rep78 protein and a second RNA sequence encoding the AVV Rep52 protein. In some embodiments, a ratio of an amount of the first RNA sequence and an amount of the second RNA sequence is at most about 1:1. In some embodiments, a ratio of an amount of AVV Rep78 protein and an amount of AVV Rep52 protein is at most about 1:1. In some embodiments, the AAV Rep protein is derived from AAV1, AAV2, AAV3, AAV4, AAV5, AAV6, AAV7, AAV8, AAV9, AAV10, AAV11, AAV12, or AAV13.

In some embodiments, the method further comprises delivering an additional RNA sequence encoding an AAV Capsid (Cap) protein to the cell. In some embodiments, the AAV Cap protein is VP1, VP2 and/or VP3. In some embodiments, the AAV Cap protein comprises VP1, VP2, and VP3. In some embodiments, the additional RNA sequence comprises a third RNA sequence encoding VP1, a fourth RNA sequence encoding VP2, a fifth RNA sequence encoding VP3. In some embodiments, a ratio of an amount of the third RNA sequence, an amount of the fourth RNA, and an amount of the fifth RNA is about 1:1:10. In some embodiments, a ratio of an amount of VP1, an amount of VP2, and an amount of VP3 is about 1:1:10. In some embodiments, the AAV Cap protein is derived from AAV1, AAV2, AAV3, AAV4, AAV5, AAV6, AAV7, AAV8, AAV9, AAV10, AAV11, AAV12, or AAV13. In some embodiments, a ratio of the amount of the first RNA sequence, the amount of the second RNA sequence, the amount of the third RNA sequence, the amount of the fourth RNA, and the amount of the fifth RNA is 1:10:5:5:50, 1:1:4:4:10, or 2:2:3:3:30. In some embodiments, a ratio of the amount of Rep78 protein, the amount of Rep52 protein, the amount of VP1, the amount of VP2, and the amount of VP3 is 1:10:5:5:50, 1:1:4:4:10, or 2:2:3:3:30.

In some embodiments, the method further comprises delivering a nucleic acid vector comprising a transgene to the cell. In some embodiments, the nucleic acid vector is a deoxyribonucleic acid (DNA) vector. In some embodiments, the nucleic acid vector comprises an inverted terminal repeat (ITR). In some embodiments, the method further comprises delivering a nucleic acid sequence encoding a helper protein or a helper RNA into the cell.

In another aspect, the present disclosure provides a method for producing recombinant adeno-associated viral (AAV) particles, comprising: (a) providing a first ribonucleic nucleic (RNA) sequence encoding at least one AAV Rep protein and a second ribonucleic nucleic (RNA) sequence encoding at least one AAV Cap protein; (b) delivering the first and the second RNA sequence into a cell; and (c) collecting the recombinant AAV particles from the cell.

In some embodiments, the at least one AAV Rep protein comprises an AVV Rep78 protein and/or an AAV Rep 52 protein. In some embodiments, the at least one AAV Cap protein comprises VP1, VP2 and/or VP3.

In another aspect, the present disclosure provides a cell for producing recombinant adeno-associated viral (AAV) particles, comprising: a ribonucleic acid (RNA) sequence encoding an AAV Rep protein, wherein the cell (i) does not comprise a deoxyribonucleic acid (DNA) sequence encoding the AAV Rep protein, or (ii) comprises a low amount of the DNA sequence encoding the AAV Rep protein wherein a concentration ratio of the DNA sequence to the RNA sequence is lower than about 0.01. In some embodiments, the cell is a mammalian cell or a non-mammalian cell.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### DETAILED DESCRIPTION OF THE INVENTION

In this disclosure, the use of the singular includes the plural unless specifically stated otherwise. Also, the use of “or” means “and/or” unless stated otherwise. Similarly, “comprise,” “comprises,” “comprising” “include,” “includes,” and “including” are not intended to be limiting.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

### Overview

The present disclosure provides compositions and methods to produce recombinant adeno-associated viral vectors for use in gene delivery. The compositions and methods provided herein can ensure that replication-competent adeno-associated viruses (AAVs) do not arise from packaging. In various aspects of the present disclosure, ribonucleic acids (e.g., messenger ribonucleic acids or mRNAs) can be used to deliver viral proteins for packaging recombinant AAV particles.

Expression level of viral proteins can be finely tuned by adjusting the concentration of RNAs (e.g., mRNAs) encoding each viral protein to maximize packaging yield. Expression can be immediate after delivery (e.g., by chemical delivery, by microinjection, or by electroporation), offering fast packaging reactions. The RNAs (e.g., mRNAs) encoding viral proteins used in the present disclosure can be modified. Various modifications can be used.

Packaging reaction may be performed with or without helper function. Although conventionally AAV packaging may be enhanced by helper function such as infection of helper virus (e.g., adenovirus, HSV), or co-transfection of helper plasmids (e.g., encoding adenovirus Ela, E1b, E2a, E4 ORF6, VA RNA, or a subset thereof), AAV can be packaged in human cells without helper function in the presence of genotoxic substances. In some cases, AAV can be packaged in insect cells by introducing rep, cap and recombinant AAV vectors (via baculoviral vector) without helper function.

Packaging cell can be eukaryotic cell, including mammalian cell or non-mammalian cell. Mammalian cell may be derived from human, mouse, rat, or hamster. Non-mammalian cell may be derived from arthropod, such as insect, such as lepidoptera. Cells may be grown in serum-free media or media free of animal-based products. RNA delivery method can be chemical transfection, microinjection, or electroporation.

An example method for producing recombinant AAV particles provided herein can comprise delivering an RNA sequence encoding an AAV Rep protein into a cell. The RNA sequence can be a messenger RNA. The AAV Rep protein can be an AVV Rep78 protein or an AAV Rep 52 protein. The AAV Rep protein can comprise AVV Rep78 protein and AAV Rep52 protein. The RNA sequence can comprise a first RNA sequence encoding the AVV Rep78 protein and a second RNA sequence encoding the AVV Rep52 protein.

The ratio of an amount of the first RNA sequence and an amount of the second RNA sequence may be at most about 1:1. The ratio of an amount of AVV Rep78 protein and an amount of AVV Rep52 protein may be at most about 1:1. The AAV Rep protein can be derived from various types of AAVs, including but not limited to, AAV1, AAV2, AAV3, AAV4, AAV5, AAV6, AAV7, AAV8, AAV9, AAV10, AAV11, AAV12, or AAV13. Next, an additional RNA sequence encoding an AAV Cap protein can be delivered into the cell. The AAV Cap protein can be VP1, VP2 and/or VP3. The AAV Cap protein can comprise VP1, VP2, and VP3. The additional RNA sequence can comprise a third RNA sequence encoding VP1, a fourth RNA sequence encoding VP2, a fifth RNA sequence encoding VP3. The ratio of an amount of the third RNA sequence, an amount of the fourth RNA, and an amount of the fifth RNA may be about 1:1:10. The ratio of an amount of VP1, an amount of VP2, and an amount of VP3 may be about 1:1:10. The AAV Cap protein can be derived from various types of AAVs, including but not limited to, AAV1, AAV2, AAV3, AAV4, AAV5, AAV6, AAV7, AAV8, AAV9, AAV10, AAV11, AAV12, or AAV13. The ratio of the amount of the first RNA sequence, the amount of the second RNA sequence, the amount of the third RNA sequence, the amount of the fourth RNA, and the amount of the fifth RNA may be 1:10:5:5:50, 1:1:4:4:10, or 2:2:3:3:30. The ratio can be adjusted experimentally. The ratio of the amount of Rep78 protein, the amount of Rep52 protein, the amount of VP1, the amount of VP2, and the amount of VP3 may be 1:10:5:5:50, 1:1:4:4:10, or 2:2:3:3:30. Next, a nucleic acid vector comprising a transgene can be delivered into the cell. The nucleic acid vector can be a DNA vector. The nucleic acid vector can comprise an inverted terminal repeat (ITR). The ITR can be derived from various types of AAVs described herein and can be of various length described herein. Optionally, a nucleic acid sequence encoding a helper protein or a helper RNA sequence can be delivered into the cell.

Another example method for producing recombinant AAV particles can comprise providing a first RNA sequence encoding at least one AAV Rep protein and a second RNA sequence encoding at least one AAV Cap protein. Next, the first and the second RNA sequence can be delivered into a cell. Next, the recombinant AAV particles can be collected from the cell.

An example composition provided herein can comprise a cell for producing recombinant AAV particles. The cell can comprise an RNA sequence encoding an AAV Rep protein. The cell (i) may not comprise a DNA sequence encoding the AAV Rep protein, or (ii) may comprise a low amount of the DNA sequence encoding the AAV Rep protein. For example, a concentration ratio of the DNA sequence to the RNA sequence may be lower than about 0.01.

### Adeno-Associated Viruses

Adeno-associated virus (AAV) is part of the genus Dependoparvovirus, which belongs to the family Parvoviridae. AAV is a small, non-enveloped, icosahedral virus with single-stranded DNA (ssDNA) genome of approximately 4.7 kilobases (kb) to 6 kb in length. Several serotypes have been discovered, with AAV serotype 2 (AAV2) as the most extensively examined serotype so far.

The AAV genome can comprise two open reading frames, rep and cap, flanked by two 145 base inverted terminal repeats (ITRs). These ITRs base pair can allow for synthesis of the complementary DNA strand. The rep and cap genes (which may also be collectively referred to as the rep/cap gene) can be translated to produce multiple distinct proteins: the rep gene can encode the proteins Rep78, Rep68, Rep52, and/or Rep40 which may function to support the AAV life cycle; the cap gene can encode VP1, VP2, and/or VP3 which are the capsid proteins. When constructing an AAV transfer plasmid (or recombinant AAV vector), the transgene can be placed between the two ITRs, and the nucleic acid sequences containing the rep and cap gene or portion thereof can be supplied in trans. The AAV particles produced by the host cell may be replication defective. The ITRs of the recombinant AAV vector can comprise a full-length ITR sequence (e.g., the full-length sequence of a wild-type ITR). The ITRs of the recombinant AAV vector can comprise a portion of the full-length ITR sequence. For example, the ITRs of the recombinant AAV vector can comprise at least about 80, 90, 100, 110, 120, 130, 140, or 145 nucleotides of the full-length ITR sequence.

The AAV rep coding sequences can encode one or more replication proteins that can be used for viral genome replication and packaging into new virions (e.g., viral particles). The rep gene can generally encode at least one large Rep protein (e.g., Rep78/68) and one small Rep protein (e.g., Rep52/40), however in some cases described herein, the rep gene may not need to encode all of the AAV Rep proteins. Therefore, in some embodiments, the Rep proteins comprise the Rep78 protein and the Rep52 and/or Rep40 proteins. In some embodiments, the Rep proteins comprise the Rep68 and the Rep52 and/or Rep40 proteins. In some embodiments, the Rep proteins comprise the Rep68 and Rep52 proteins, Rep68 and Rep40 proteins, Rep78 and Rep52 proteins, or Rep78 and Rep40 proteins. In some embodiments, the Rep proteins comprise the Rep78, Rep68, Rep52 and Rep40 proteins. In some embodiments, the Rep proteins comprise the Rep78 protein, the Rep68 protein, the Rep52 protein, or the Rep40 protein, or any combinations thereof.

The AAV cap coding sequences can encode the structural proteins that form a functional AAV capsid. For example, the structural proteins can package DNA and infect host cells. The cap coding sequences can encode at least one or all of the AAV capsid subunits, but less than all of the capsid subunits may be encoded or expressed as long as a functional capsid can be produced. In some embodiments, the Cap proteins encoded comprise VP1, VP2 and/or VP3. In some embodiments, the Cap proteins encoded comprise VP1, VP2, or VP3, or any combinations thereof.

The coding sequence described herein can be RNA sequences. In various embodiments, a mixture of RNA sequences encoding Rep proteins and/or Cap proteins are produced in vitro, for example, by in vitro transcription. The mixture can be delivered into a host cell for viral packaging. The mixture can comprise RNA sequences encoding Rep proteins and/or Cap proteins mixed at different ratios. For example, the ratio of Rep78:Rep52, which represents ratio of the amount of corresponding RNAs, can be approximately 1:10. The ratio of the amount can represent the mass ratio of the corresponding RNAs. The proteins encoded by such ratio can have similar ratios of the protein amount, and in some cases, the ratio of Rep78:Rep52 (or VP1:VP2:VP3) can also indicate the ratio of the encoded proteins. In some cases, the ratio of Rep78:Rep52 can be at most about 1:1, 1:2, 1:3, 1:4, 1:5, 1:6, 1:7, 1:8, 1:9, 1:10, 1:11, 1:12, 1:13, 1:14, 1:15 or less. In some cases, the ratio of Rep78:Rep52 can be at least about 1:30, 1:25, 1:20, 1:15, 1:10, 1:9, 1:8, 1:7, 1:6, 1:5 or more. The ratio of VP1:VP2:VP3 can be approximately 1:1:10. The ratio of VP1:VP2:VP3 can be approximately 1:1:2, 1:1:3, 1:1:4, 1:1:5, 1:1:6, 1:1:7, 1:1:8, 1:1:9, 1:1:10, 1:1:11, 1:1:12, 1:1:13, 1:1:14, 1:1:15, 1:1:16, 1:1:17, 1:1:18, 1:1:19, 1:1:20, 1:2:2, 1:2:3, 1:2:4, 1:2:5, 1:2:6, 1:2:7, 1:2:8, 1:2:9, 1:2:10, 1:2:11, 1:2:12, 1:2:13, 1:2:14, 1:2:15, 1:2:16, 1:2:17, 1:2:18, 1:2:19 or 1:2:20. In some cases, the ratio of VP1:VP2 can be at least about 1:10, 1:9, 1:8, 1:7, 1:6, 1:5, 1:4, 1:3, 1:2, 1:1, 2:1, 2.5:1, 3:1, 3.5:1, 4:1, 4.5:1, 5:1, 5.5:1, 6:1 or more. In some cases, the ratio of VP1:VP2 can be at most about 10:1, 9:1, 8:1, 7:1, 6:1, 5:1, 4:1, 3:1, 2:1, 1:1, 1:2, 1:2.5, 1:3, 1:3.5, 1:4, 1:4.5, 1:5, 1:5.5, 1:6 or less. In some cases, the ratio of VP1:VP3 can be at least about 1:10, 1:9, 1:8, 1:7, 1:6, 1:5, 1:4, 1:3, 1:2, 1:1, 2:1, 2.5:1, 3:1, 3.5:1, 4:1, 4.5:1, 5:1, 5.5:1, 6:1 or more. In some cases, the ratio of VP1:VP3 can be at most about 10:1, 9:1, 8:1, 7:1, 6:1, 5:1, 4:1, 3:1, 2:1, 1:1, 1:2, 1:2.5, 1:3, 1:3.5, 1:4, 1:4.5, 1:5, 1:5.5, 1:6 or less. In some cases, the ratio of VP2:VP3 can be at least about 1:10, 1:9, 1:8, 1:7, 1:6, 1:5, 1:4, 1:3, 1:2, 1:1, 2:1, 2.5:1, 3:1, 3.5:1, 4:1, 4.5:1, 5:1, 5.5:1, 6:1 or more. In some cases, the ratio of VP2:VP3 can be at most about 10:1, 9:1, 8:1, 7:1, 6:1, 5:1, 4:1, 3:1, 2:1, 1:1, 1:2, 1:2.5, 1:3, 1:3.5, 1:4, 1:4.5, 1:5, 1:5.5, 1:6 or less. The amount of total RNAs encoding the Cap/VP proteins may be higher than the amount of total RNAs encoding Rep proteins. For example, the amount of total RNAs encoding the Cap/VP proteins may be at least about 1.5, 2, 2.5, 3, 3.5, 4, 4.5, 5, 5.5, 6, 6.5, 7, 7.5, 8, 8.5, 9, 9.5, 10 or more times the amount of total RNAs encoding Rep proteins. In some cases, the ratio of Rep78:VP1 can be at least about 1:10, 1:9, 1:8, 1:7, 1:6, 1:5, 1:4, 1:3, 1:2, 1:1, 2:1, 2.5:1, 3:1, 3.5:1, 4:1, 4.5:1, 5:1, 5.5:1, 6:1 or more. In some cases, the ratio of Rep78:VP1 can be at least about 2:3, 2.5:3, 3.5:3, 4:3 or more. In some cases, the ratio of Rep78:Rep52:VP1:VP2:VP3 can be approximately 1:10:5:5:50. In some cases, the ratio of Rep78:Rep52:VP1:VP2:VP3 can be approximately 1:1:4:4:10. In some cases, the ratio of Rep78:Rep52:VP1:VP2:VP3 can be approximately 2:2:3:3:30. To optimize the mRNA ratio and concentration, a well-established packaging plasmid such as commercially available pRepCap (a plasmid used to express the Rep proteins and Cap/VP proteins in AAV packaging) can be used to transfect the packaging cell line. In parallel, the mRNA cocktail can be used to transfect the same packaging cell line. Western blot can be used to quantify the Rep78, Rep52, VP1, VP2, VP3 protein products in both samples. The mRNA ratio and total concentration can be adjusted accordingly so that the protein products from the mRNA transfection matches those from the plasmid transfection.

In various embodiments, a deoxyribonucleic acid (DNA) sequence encoding a viral protein may not be delivered into a cell for viral packaging. In some cases, a low amount of the DNA sequence encoding an AAV viral protein (e.g., rep or cap proteins) may be present in a host cell. In some cases, the host cell comprises an RNA sequence encoding a viral protein and a DNA sequence encoding the viral protein, where a concentration ratio of the DNA sequence to the RNA sequence is lower than or equal to about 0.1, 0.01, or 0.001. In some cases, the host cell may not comprise any DNA sequences that encode any Rep or Cap proteins described herein. Real-time PCR with and without reverse transcription can be used to quantify the ratios between the DNA sequences of interest and the RNA sequences of interest. For example, the total amount of DNA sequences encoding a given viral protein can be quantified by real-time PCR in the absence of reverse transcription, and the total amount of DNA and RNA sequences encoding a given viral protein can be quantified by real-time PCR after reverse transcription to convert the RNA sequences into DNA sequences. Then the ratios between the DNA sequences and the RNA sequences can be deduced from the above data.

The AAV ITR sequences can comprise 145 bases each and can be the cis-acting elements used for AAV genome replication and packaging into the capsid. The ITR sequences can be at the 5′ and 3′ ends of the vector genome and flank the heterologous nucleic acid sequence of interest (e.g., a transgene), but may not be contiguous thereto. The ITR sequences can be the same or different from each other. For example, one of the ITR sequence can be derived from a first AAV serotype, and the other ITR sequence can be derived from a second AAV serotype that is different from the first AAV serotype. For another example, one of the ITR sequences can have a length that is different from the other ITR sequence.

References to AAV as used herein, includes, but is not limited to, AAV serotype 1 (AAV1), AAV serotype 2 (AAV2), AAV serotype 3 (including serotypes 3A and 3B) (AAV3), AAV serotype 4 (AAV4), AAV serotype 5 (AAV5), AAV serotype 6 (AAV6), AAV serotype 7 (AAV7), AAV serotype 8 (AAV8), AAV serotype 9 (AAV9), AAV serotype 10 (AAV10), AAV serotype 11 (AAV11), AAV serotype 12 (AAV12), AAV serotype 13 (AAV13), snake AAV, avian AAV, bovine AAV, canine AAV, equine AAV, ovine AAV, goat AAV, shrimp AAV, and any other AAV now known or later discovered.

References to AAV may include artificial AAV serotypes which include, without limitation, AAV with a non-naturally occurring capsid protein. Such an artificial capsid may be generated by any suitable technique, using one AAV serotype sequence (e.g., a fragment of a VP1 capsid protein) in combination with heterologous sequences which may be obtained from another AAV serotype, non-contiguous portions of the same AAV serotype, from a non-AAV viral source, or from a non-viral source. An artificial AAV serotype may be, without limitation, a chimeric AAV capsid, a recombinant AAV capsid, or a “humanized” AAV capsid.

An AAV ITR may be from any AAV, including but not limited to serotypes 1, 2, 3a, 3b, 4, 5, 6, 7, 8, 9, 10, 11, or 13, snake AAV, avian AAV, bovine AAV, canine AAV, equine AAV, ovine AAV, goat AAV, shrimp AAV, or any other AAV. An AAV ITR may not have the native terminal repeat sequence (e.g., a native AAV ITR sequence may be altered by insertion, deletion, truncation and/or missense mutations), as long as the terminal repeat mediates the desired functions, e.g., replication, virus packaging, and/or integration, and the like.

The nucleic acid sequences (e.g., RNAs) encoding the Rep or Cap protein can be derived from AAV1, AAV2, AAV3, AAV4, AAV5, AAV6, AAV7, AAV8, AAV9, AAV10, AAV11, AAV12, AAV13 or any combinations thereof. In some embodiments, the nucleic acid sequences (e.g., RNAs) encoding the Rep or Cap protein can be derived from AAV2, AAV5 and/or AAV9. The nucleic acid sequences encoding the Rep or Cap protein can be derived from snake AAV, avian AAV, bovine AAV, canine AAV, equine AAV, ovine AAV, goat AAV, shrimp AAV, or an artificial AAV.

Alternatively, in some embodiments, the rep coding sequences encoding the Rep proteins are from an AAV serotype which differs from that which is providing the cap coding sequences encoding the Cap proteins. Therefore, in some embodiments, the rep coding sequences are fused in frame to cap coding sequences of a different AAV serotype to form a chimeric AAV vector. For example, the rep coding sequence can be derived from AAV2 and the cap coding sequence can be derived from AAV2 or AAV5 to produce AAV2-like and AAV5-like particles, respectively. These may be named rep2cap2 and rep2cap5.

The genomic sequences of various serotypes of AAV, as well as the sequences of the native ITRs, Rep proteins, and capsid subunits are known. Such sequences may be found in the literature or in public databases such as GenBank. See, e.g., GenBank Accession Numbers NC_002077, NC_001401, NC_001729, NC_001863, NC_001829, NC_001862, NC_000883, NC_001701, NC_001510, NC_006152, NC_006261, AF063497, U89790, AF043303, AF028705, AF028704, J02275, J01901, J02275, X01457, AF288061, AH009962, AY028226, AY028223, AY631966, AX753250, EU285562, NC_001358, NC_001540, AF513851, AF513852 and AY530579; the disclosures of which are incorporated by reference herein for AAV nucleic acid and amino acid sequences.

### Helper Proteins or Sequences

In addition to Rep and Cap proteins, in some cases, AAV may use a helper virus or plasmid containing genes for AAV replication because AAV may not have the ability to replicate on its own. In the absence of helper viruses, AAVs may incorporate into the host cell genome, for example, at a specific site of chromosome 19. The helper viruses can support a productive AAV infection. These viruses include, but are not limited to, adenovirus, herpes virus, cytomegalovirus, Epstein-Barr virus and vaccinia virus. The helper virus vectors of the present disclosure can comprise DNA from a helper virus, which DNA provides for the helper-viral functions useful for a productive AAV infection, but the vector itself may not be packaged into infectious helper virus virions. A helper virus vector may contain the entire genomic DNA of the helper virus except for the cis-acting signals that function in the replication and/or packaging of the helper virus. Helper virus sequences containing genes for AAV replication can be provided by a helper adenovirus or herpesvirus vector. The helper virus genes can encode proteins (e.g., helper proteins) and/or non-coding RNA (e.g., helper RNA). In the present disclosure, nucleic acid sequences encoding helper proteins can be prepared and delivered into a host cell. The nucleic acid sequences encoding helper proteins can be DNA or RNA.

In some embodiments, the DNA or RNA sequences encoding helper proteins are derived from adenovirus. In some embodiments, the adenovirus is selected from adenovirus 2 and adenovirus 5.

In some embodiments, the DNA or RNA sequences comprise all or part of E4, E2a and VA genes derived from adenovirus, in particular adenovirus 2. In some cases, not all of the native adenovirus genes may be required for AAV replication, for example only the E4 34 kD protein encoded by open reading frame 6 (ORF6) of the E4 gene may be used for AAV replication. Therefore, in some embodiments, the DNA or RNA sequences comprise an E4 ORF6 coding region, an adenovirus E2a 72 kD coding region (coding for the E2a 72 kD DNA-binding protein) and a VA RNA. In some embodiments, the DNA or RNA sequences additionally comprise sequences encoding adenovirus Ela and E1b. In some embodiments, the DNA or RNA sequences encoding helper proteins comprise sequences encoding adenovirus Ela, E1b, E2a, E4 ORF6, or VA RNA, or a subset thereof.

In some embodiments, the DNA or RNA sequences encoding helper proteins are derived from herpesvirus. In some embodiments, the herpesvirus is selected from: herpes simplex virus (HSV), Epstein-Barr Virus (EBV), cytomegalovirus (CMV) and pseudorabies virus (PRV).

In some cases, a helper protein may not be needed for viral packaging and in such cases, and in such cases, the DNA or RNA sequences encoding helper proteins may not be delivered into the host cell for packaging viral particles. For example, Sf9 cell can support viral packaging without the helper function.

### Nucleic Acid Vectors

The present disclosure provides methods to produce recombinant AAV particles in a cell. In various embodiments, a nucleic acid vector comprising a transgene is delivered to the cell. The nucleic acid vector can comprise the transgene encoded between the two AAV ITRs, optionally including a promoter and/or a polyA signal. The nucleic acid vector can be a deoxyribonucleic acid (DNA) vector. The nucleic acid vector can be single-stranded or double-stranded. The nucleic acid vector can be a plasmid. The nucleic acid vector can be linear or circular.

The nucleic acid vector can be delivered into the cell together with one or more RNA sequences encoding one or more viral proteins for packaging recombinant AAV particles comprising the transgene.

The transgene may be a therapeutically active gene which encodes a gene product which may be used to treat or ameliorate a disease. This may include, for example, when a target gene is not expressed correctly in the host cell, therefore a corrected version of the target gene is introduced as the transgene. Therefore, the transgene may be a gene of potential therapeutic interest. The transgene may have been obtained from another cell type, or another species, or prepared synthetically. Alternatively, the transgene may have been obtained from the host cell, but operably linked to regulatory regions which are different to those present in the native gene. Alternatively, the transgene may be a different allele or variant of a gene present in the host cell.

The transgene may encode, for example, an antisense RNA, a ribozyme, a protein (for example, a subunit of a T cell receptor), a toxin, an antigen (which may be used to induce antibodies or helper T cells or cytotoxic T cells) or an antibody (such as a single chain antibody). Any gene that is flanked by the ITRs can effectively be packaged into an AAV capsid. In some embodiments, the transgene is less than or equal to about 5, 4.5, 4, 3.5, 3, 2.5, 2, 1.5 or 1 kb long.

Multiple copies of the transfer plasmid containing the transgene may result in higher transgene production, therefore in some embodiments, the nucleic acid vector comprises multiple copies of the transgene, such as two or more, or three or more, copies of the transgene. In some embodiments, the nucleic acid vector comprises at least 2, 3, 4, 5, 6, 7, 8, 9, 10 or more copies of the transgene.

In some cases, more than one gene product may be required to treat a disease, therefore in some embodiments, the nucleic acid vector can additionally comprise two or more, three or more, or four or more, different transgenes. In some cases, the nucleic acid vector can comprise at least 2, 3, 4, 5, 6, 7, 8, 9, 10 or more different transgenes.

Gene therapy can be used to modify the genetic material of living cells for therapeutic purposes, and it may involve the insertion of a functional gene into a cell to achieve a therapeutic effect. The recombinant AAV vector produced using the compositions and methods described herein can be used to transduce target cells and induce the expression of the gene of potential therapeutic interest. The recombinant AAV vector can therefore be used for treatment of a mammalian subject, such as a human subject, suffering from a condition including but not limited to, inherited disorders, cancer, and certain viral infections.

The transgene can encode any polypeptide or RNA that can be produced in a cell in vitro, ex vivo, or in vivo. For example, the recombinant AAV vectors may be introduced into cultured cells and the expressed gene product can be isolated therefrom.

### Host Cells

The present disclosure provides methods to produce ribonucleic acid (RNA) sequences encoding viral proteins and methods to deliver the RNA sequences into a host cell for packaging recombinant adeno-associated viral particles. The host cell can be referred to as a packaging cell in the present disclosure. The host cell can be a eukaryotic cell. The host cell can be a mammalian cell. The host cell can be a human cell. In some embodiments, the mammalian cell can be selected from a HEK 293 cell, CHO cell, Jurkat cell, KS62 cell, PerC6 cell, HeLa cell or a derivative or functional equivalent thereof. In some embodiments, the mammalian host cell is a HEK 293 cell, or derived from a HEK 293 cell. Such cells could be adherent cell lines (e.g., they grow in a single layer attached to a surface) or suspension adapted/non-adherent cell lines (e.g., they grow in suspension in a culture medium). The HEK 293 cell can be a HEK 293T cell. The term “HEK 293 cell” refers to the Human Embryonic Kidney 293 cell line which is commonly used in biotechnology. In some cases, HEK 293 cells can be used for the production of AAV vectors because they already contain the E1A and E1B helper virus genes, so only the E2A, E4 ORF6 and VA helper factors may be provided. Other examples of suitable commercially available cell lines include T-REX™ (Life Technologies) cell lines.

The host cell can be a non-mammalian cell. For example, the host cell can be an insect cell such as Sf9 cell line. The AAV particles can be packaged in insect cells by introducing rep, cap and nucleic acid vector containing a transgene (via baculoviral vector) without helper function.

### Ribonucleic Acid Sequences and Modifications

The present disclosure provides methods to produce and use RNA sequences encoding AAV viral proteins for recombinant AAV packaging. The RNA molecules described herein can be modified. The modified RNA molecule may comprise at least one modification and a translatable region. The modified RNA molecule can comprise at least two modifications and a translatable region. The modified RNA molecule can comprise at least 2, 3, 4, 5, 6, 7, 8, 9, 10 or more modifications. The modification may be located on the backbone and/or a nucleoside of the RNA molecule. The modification may be located on both a nucleoside and a backbone linkage.

A modification may be located on the backbone linkage of the modified RNA molecule. The backbone linkage may be modified by replacing of one or more oxygen atoms. The modification of the backbone linkage may comprise replacing at least one phosphodiester linkage with a phosphorothioate linkage.

A modification may be located on a nucleoside of the modified RNA molecule. The modification on the nucleoside may be located on the sugar of the nucleoside. The modification of the nucleoside may occur at the 2′ position on the nucleoside.

The nucleoside modification may include a compound selected from the group consisting of pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine. The modifications can be independently selected from the group consisting of 5-methylcytosine, pseudouridine and 1-methylpseudouridine.

A modification may be located on a nucleobase of the modified RNA molecule. The modification on the nucleobase may be selected from the group consisting of cytosine, guanine, adenine, thymine and uracil. The modification on the nucleobase may be selected from the group consisting of deaza-adenosine and deaza-guanosine, and the linker may be attached at a C-7 or C-8 position of said deaza-adenosine or deaza-guanosine. The modified nucleobase may be selected from the group consisting of cytosine and uracil, and the linker may be attached to the modified nucleobase at an N-3 or C-5 position. The linker attached to the nucleobase may be selected from the group consisting of diethylene glycol, dipropylene glycol, triethylene glycol, tripropylene glycol, tetraethylene glycol, tetraethylene glycol, divalent alkyl, alkenyl, alkynyl moiety, ester, amide, and ether moiety.

In some embodiments, two modifications of the RNA molecule may be located on nucleosides of the modified RNA molecule. The modified nucleosides may be selected from 5-methylcytosine and pseudouridine.

In some embodiments, two modifications of the modified RNA molecule may be located on a nucleotide or a nucleoside. The RNA molecule may comprise a polyA tail of at least about 5, 10, 15, 20, 25, 30, 35, 40, 45, 50, 55, 60, 65, 70, 75, 80, 85, 90, 95, 100, 120, 150, 160, 200, or more nucleotides in length. Further, the nucleic acid molecule may comprise at least one 5′ terminal cap such as, but not limited to, Cap0, Cap1, ARCA, inosine, N1-methyl-guanosine, 2′ fluoro-guanosine, 7-deaza-guanosine, 8-oxo-guanosine, 2-amino-guanosine, LNA-guanosine, and 2-azido-guanosine. The RNA molecule provided herein can further comprise 5′ and/or 3′ untranslated regions. The RNA molecule may also comprise an internal ribosome entry site (IRES).

### EXAMPLES

### Example 1: Packaging Recombinant AAV in 293 or Sf9 Cells

DNA comprising the complete, intron-free coding sequence of Rep78, Rep52 of AAV2 and VP1, VP2 and VP3 of a particular serotype of AAV can be synthesized. Optionally, coding sequence of adenovirus E1a, E1b, E2a, E4 (or more specifically, E4 ORF6), as well as the exact sequence of VA RNA can be synthesized. A T7 promoter can be appended upstream of these DNA so that the mRNA can be transcribed by T7 RNA Polymerase in vitro. The in vitro transcription (IVT) reaction may contain capped G as initiator so that the mRNA is capped for eukaryotic translation. Alternatively, the mRNA can be enzymatically capped. Each mRNA can be purified and concentrated to 1 to 5 mg/mL. A cocktail of mRNA can be mixed where the ratio of Rep78:Rep52:VP1:VP2:VP3 can be approximately 1:10:5:5:50. For a new cell line or a new serotype, this may serve as a starting ratio since it has been shown that (1) a high Rep52:Rep78 ratio may be beneficial for AAV yield, (2) the natural ratio of VP1:VP2:VP3 protein is approximately 1:1:10, and (3) the overall expression level of rep proteins may be lower than that of Cap/VP proteins. However, due to the potential difference in transfection efficiency and/or translation efficiency, the exact ratio among different mRNAs may be further adjusted. The cocktail of mRNA may be transfected into the packaging cell line by chemical transfection, microinjection, or electroporation-based transfection.

To optimize the mRNA ratio and concentration, a well-established packaging plasmid such as commercially available pRepCap (a plasmid used to express the rep proteins and cap/VP proteins in AAV packaging) can be used to transfect the packaging cell line. In parallel, the mRNA cocktail can be used to transfect the same packaging cell line. Western blot can be used to quantify the Rep78, Rep52, VP1, VP2, VP3 protein products in both samples. The mRNA ratio and total concentration can be adjusted accordingly so that the protein products from the mRNA transfection matches those from the plasmid transfection.

The HEK 293 cells can express helper protein E1, so expressing or delivering E2a, E4, and VA RNA to HEK 293 cells may be sufficient to provide helper function. Packaging plasmids that encode these genes are readily available. So the same strategy described above can be used to determine the ratio and total concentration of these RNA species, except that the amount of VA RNA may be assessed with qRT-PCR or Northern blotting rather than Western blotting. The insect cells such as Sf9 can support AAV packaging without helper function, so no or very low amount of RNA species for helper genes may be needed.

Once the ratio and total concentration of the RNA species are determined. These RNA species may be co-transfected into the packaging cell line with a DNA comprising the transgene sequence (e.g., gene of interest to be expressed in the target cell) flanked by the inverted terminal repeats (ITRs) of AAV. The DNA may be in the form of a plasmid.

### Example 2: Packaging Recombinant AAV in 293 Cells

For production of AAV, the steps can include package cell expansion, plasmids production and transfection, viral particle production and purification. In an AAV helper-free system, rep and cap genes can be removed from the viral vector that contains AAV-2 ITRs and can be supplied in a separate plasmid called pAAV-RC. The adenoviral genes used for proper AAV packaging can be provided in the pHelper plasmid (e.g., encoding E2A, E4 and VA RNA) or in the 293 packaging cells (e.g., encoding E1). Transient expression of AAV by co-transfection of the three plasmids in either adherent or suspension HEK 293 cell lines can be used to AAV packaging.

This example shows a method to potentially increase efficiency of the initial transfection and AAV6 production by introducing sequences encoding Rep and Cap proteins as mRNA during transfection alongside a “helper” plasmid (which aids in the correct assembly of the AAV particle) and the viral vector containing ITR and the gene of interest. For producing AAV using Rep and Cap as form of mRNA, pAAV-RC plasmid and pHelper may not be used, decreasing the amount of plasmid and potential recombinant of wild-type AAV particles.

The ratio of the mRNA molecules encoding different Rep and Cap proteins were optimized during the transfection process. Two different mixtures (e.g., mix A and mix B) with different ratios were tested. The ratio of mix A was Rep78:Rep52:VP1:VP2:VP3=1:1:4:4:10. The ratio of mix B was Rep78:Rep52:VP1:VP2:VP3=2:2:3:3:30. After transfection, the virus lysate was collected, diluted and used as template in qPCR reactions for titration. The virus lysate was diluted three times with dilution factor of 50, 2, and 10. The reaction without template (e.g., no template control or NTC) was used as a control. The experimental results showed that the highest titer of AAV particles was received using Rep78:Rep52:VP1:VP2:VP3=1:1:4:4:10 (Table A). The ratio described herein is the ratio of the amount of the corresponding RNA molecules. The AAV titer was calculated in terms of genome copies per microliter (gc/4) by qPCR against a standard curve of AAV vector. The AAV production was repeated by transfecting the optimized ratio Rep/Cap and viral vector on adherent HEK293 cells and the experimental results were shown in Table B.

TABLE A
AAV6 production by suspension HEK 293 cells

| Sample | Mix A | Mix B | NTC |
|---|---|---|---|
| Cq | 27.41 | 24.04 | 31.01 |
| Copy number | 73.21 | 699.54 | |
| Copy number * | 7.32 × 10⁴ | 7 × 10⁵ | |
| Dilution factor | | | |
| (e.g., *50*2*10) | | | |
| (gc/μL) | | | |
| Total amount | 1.83 × 10⁷ | 1.05 × 10⁷ | |
| (genome copies) | | | |

TABLE B
AAV6 production by adherent HEK 293 cells

| Sample | Mix A | NTC |
|---|---|---|
| Cq | 22.72 | 32.48 |
| Copy number | 808.34 | |
| Copy number * | 8.08 × 10⁵ | |
| Dilution factor | | |
| (e.g., *50*2*10) | | |
| (gc/μL) | | |
| Total amount | 4.85 × 10⁷ | |
| (genome copies) | | |

While various embodiments of the invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1.-26. (canceled)

27. A method for producing recombinant adeno-associated viral (AAV) particles, the method comprising delivering a ribonucleic acid (RNA) sequence encoding an AAV Rep protein into a cell.

28. The method of claim 27, wherein the RNA sequence is a messenger RNA.

29. The method of claim 27, wherein the AAV Rep protein comprises an AVV Rep78 protein, an AAV Rep 68 protein, an AAV Rep 52 protein, an AAV Rep 40 protein, or any combination thereof.

30. The method of claim 29, wherein the RNA sequence comprises a first RNA sequence encoding the AVV Rep78 protein and a second RNA sequence encoding the AVV Rep52 protein, and wherein a ratio of an amount of the first RNA sequence and an amount of the second RNA sequence is at most 1:5.

31. The method of claim 27, wherein the cell (i) does not comprise a deoxyribonucleic acid (DNA) sequence encoding the AAV Rep protein, or (ii) comprises a low amount of the DNA sequence encoding the AAV Rep protein wherein a concentration ratio of the DNA sequence to the RNA sequence is lower than or equal to 0.1.

32. The method of claim 27, wherein the AAV Rep protein is from AAV1, AAV2, AAV3, AAV4, AAV5, AAV6, AAV7, AAV8, AAV9, AAV10, AAV11, AAV12, or AAV13.

33. The method of claim 30, further comprising delivering an additional RNA sequence encoding an AAV Cap protein into the cell.

34. The method of claim 33, further comprising, prior to delivering, optimizing a ratio of an amount of the RNA sequence encoding the Rep protein and the amount of the RNA sequence encoding the AAV Cap protein.

35. The method of claim 33, wherein the AAV Cap protein comprises a VP1 protein, a VP2 protein and a VP3 protein, and wherein the additional RNA sequence comprises a third RNA sequence encoding the VP1 protein, a fourth RNA sequence encoding the VP2 protein, and a fifth RNA sequence encoding the VP3 protein.

36. The method of claim 35, wherein a ratio of an amount of the third RNA sequence to an amount of the fourth RNA sequence is 1:1, a ratio of an amount of the third RNA sequence to an amount of the fifth RNA sequence is 1:10, and a ratio of an amount of the fourth RNA sequence to an amount of the fifth RNA sequence is 1:10.

37. The method of claim 35, wherein a ratio of an amount of the first RNA sequence to an amount of the third RNA sequence is at least 1:10.

38. The method of claim 33, wherein the AAV Cap protein is from AAV1, AAV2, AAV3, AAV4, AAV5, AAV6, AAV7, AAV8, AAV9, AAV10, AAV11, AAV12, or AAV13.

39. The method of claim 35, wherein a ratio of the amount of the first RNA sequence, the amount of the second RNA sequence, the amount of the third RNA sequence, the amount of the fourth RNA, and the amount of the fifth RNA is 1:10:5:5:50, 1:1:4:4:10, or 2:2:3:3:30.

40. The method of claim 27, wherein the RNA sequence comprises a modified backbone or a modified nucleoside.

41. The method of claim 27, further comprising delivering a nucleic acid vector comprising a transgene into the cell.

42. The method of claim 41, wherein the nucleic acid vector is a deoxyribonucleic acid (DNA) vector.

43. The method of claim 41, wherein the nucleic acid vector comprises an inverted terminal repeat (ITR).

44. The method of claim 27, further comprising delivering a nucleic acid sequence encoding a helper protein or a helper RNA into the cell.

45. The method of claim 27, wherein replication-competent AAV particles are not produced in the cell.

46. A cell for producing recombinant adeno-associated viral (AAV) particles, comprising: a ribonucleic acid (RNA) sequence encoding an AAV Rep protein, wherein the cell (i) does not comprise a deoxyribonucleic acid (DNA) sequence encoding the AAV Rep protein, or (ii) comprises a low amount of the DNA sequence encoding the AAV Rep protein wherein a concentration ratio of the DNA sequence to the RNA sequence is lower than about 0.01.
